(19) United States
(12) Patent Application Publication
(10) Pub. No.: US 2009/0088609 A1
(43) Pub. Date: Apr. 2, 2009

(54) Method of taking internal physiological measurements

(76) Inventors: Thomas Schmitz-Rode, Aachen (DE); Joachim Georg Pfeffer, Aachen (DE); Karl Michael Ruhl, Simmerath (DE); Uwe Schnakenberg, Aachen (DE)

Correspondence Address: K.F. ROSS P.C., 5683 RIVERDALE AVENUE, SUITE 203 BOX 900, BRONX, NY, 10471-0900, US

(21) Appl. No.: 11/919,774

(22) PCT Filed: Apr. 28, 2006
(86) PCT No.: PCT/EP2006/004001

§ 371 Date: Jul. 18, 2008

(30) Foreign Application Priority Data
May 3, 2005 (DE) 10 2005 020 980.7
Jul. 27, 2005 (DE) 10 2005 035 795.4

(51) Int. Cl. A61B 5/00; A61M 25/00

(52) U.S. Cl. 600/301; 604/264

(57) ABSTRACT

The invention relates to a device for detecting physiological measured variables within the body, comprising a catheter which has a tip supporting a sensor and which can be introduced into a blood vessel, the catheter (1) being connected to a transponder unit (2) for wireless transmission of at least one measured variable to an external reading station which is arranged outside the blood vessel (4) after insertion of the catheter (1) into said blood vessel (4), the catheter (1) and the transponder (2) being sealed in a leaktight manner. The invention also relates to a sluice/sleeve for coaxial arrangement on an element for insertion into a blood vessel, in particular a needle and/or a catheter, the sluice/sleeve (6,10,11) being able to be divided at least once in the longitudinal direction.

The invention relates to a device for measuring physiological variables within the body, comprising a catheter having a tip that is insertable into a blood vessel and that carries a sensor.

It is known from the prior art, e.g. from EP 0 646 350 [U.S. Pat. No. 7,263,894], to puncture a blood vessel with a catheter, so that it is possible to measure via the catheter tip the pressure within the lumen of the blood vessel. To this end, the catheter tip is connected to a pressure-measuring instrument that during catheterization lies outside the body of the patient being treated. This procedure is therefore an invasive examination measure in which the catheter projecting from the body represents an entry point for germs. In addition, catheters are known that have a sensor at their tip in order to monitor physiological variables, in particular, intravascular measured variables.

It is a fundamental problem that a catheter must be inserted through a puncture in vessel wall into the lumen of the vessel. In particular, when an artery is punctured, the wall trauma, and thus bleeding, must be minimized. A known approach in this regard is to use sheaths as well as hemostatic agents when inserting a catheter into the blood vessel.

Even when an extremely thin sheath for catheter insertion in the blood vessel is removed, it is impossible to prevent bleeding. Also, with an ambulatory implantable catheter, fast and lasting bleeding control is extremely important since the puncture orifice in the vessel wall is always somewhat larger than the subsequently inserted catheter.

Thrombus deposits on the catheter are a problem. They must be avoided since they can result in embolisms downstream in the vessel and, if the sensor surface is covered, can result in faulty measurements, or in the worst case the total blockage of the blood vessel.

In addition, the procedure must ensure a sufficient implantation depth of the catheter in the blood vessel, and an orientation must be provided for the tester in this regard. Any displacement of the catheter, e.g. by forces or relative movements on the catheter section not located in the blood vessel caused by muscle or joint movements, must be prevented. In the worst case, the catheter's slipping out of the blood vessel could possibly result in life-threatening bleeding.

The sensor must be placed such that during a pressure measurement only the static pressure is detected. The additive measurement of back pressure must be precluded.

Known catheters also have the disadvantage that the end of the catheter lies outside the body and thus provides an entryway for germs, thereby making long-term monitoring impossible. In particular, a patient is not able to wear this type of catheter arrangement on a sustained basis.

The goal of the invention is therefore to overcome the above-referenced disadvantages of the prior art, and in particular, to create a catheter or a catheter system by means of which wall trauma can be minimized when an artery is punctured and bleeding can be prevented. In addition, it must be possible to implement fast and permanent hemostasis, even if the puncture orifice in the vessel wall is somewhat larger than the inserted or implantable catheter. Sheaths for introducing the catheter into the vessel should be removable without the cable or catheter connection having to be disengaged.

In terms of further goals, thrombus deposits on the catheter should be precluded, a sufficient implantation depth of the catheter in the blood vessel should be achieved, and an orientation in this regard should be provided to the technician. It is essential that displacement of the catheter be prevented and the sensor should be positioned such that during pressure measurement only static pressure is detected and any additive measurement of back pressure is precluded.

According to the invention, these objects are attained by a catheter that is connected to a transponder unit or a telemetry unit for wirelessly transmitting at least one measured variable to an external reader, whereby after insertion of the catheter the transponder unit is outside the blood vessel, i.e. extravasally in the adjacent tissue, the catheter and transponder being coupled and sealed in a fluid-tight manner. In addition, this object is attained by a sheath/sleeve, in particular for use with such a catheter, the sheath/sleeve being easily split at least longitudinally, i.e. such that each of the possible divisions run longitudinally.

Due to the fact that the catheter and transponder are sealed in a fluid-tight manner and form one unit, this arrangement is able to remain within the body of the patient even for prolonged tests. Any desired measured variables can be detected by the sensor in the tip of the catheter and transmitted by the transponder to a reader located outside the body. For this purpose, conventional RFID technology, for example, can be employed.

By way of example and without restricting the invention, application in a blood-pressure measuring system is described based on the following figures: Therein:

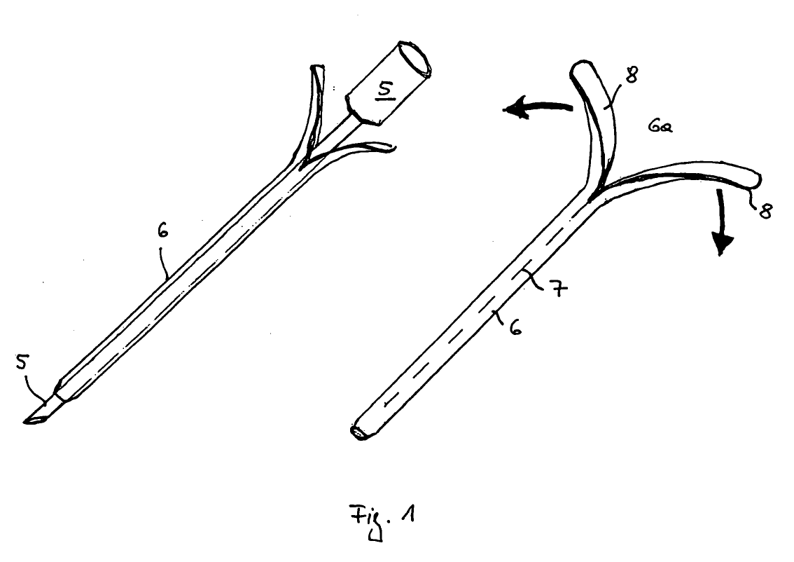

FIG. 1 shows a sheath according to the invention with and without puncture needle;

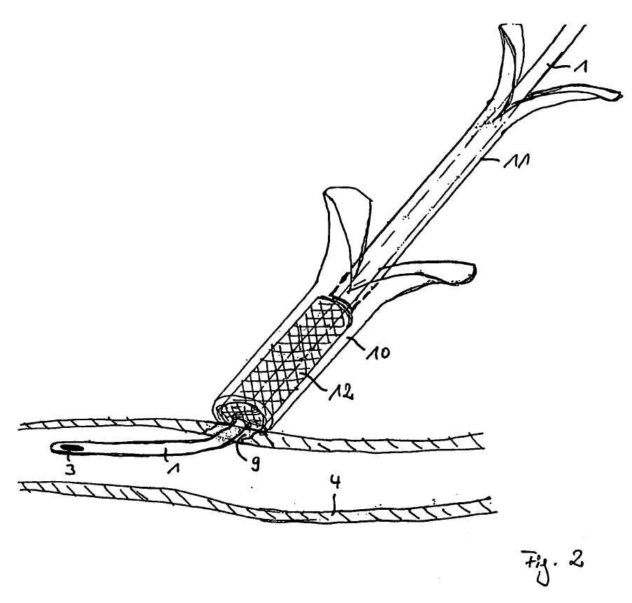

FIG. 2 shows a catheter according to the invention after insertion into a blood vessel and having ancillary elements coaxially surrounding the catheter;

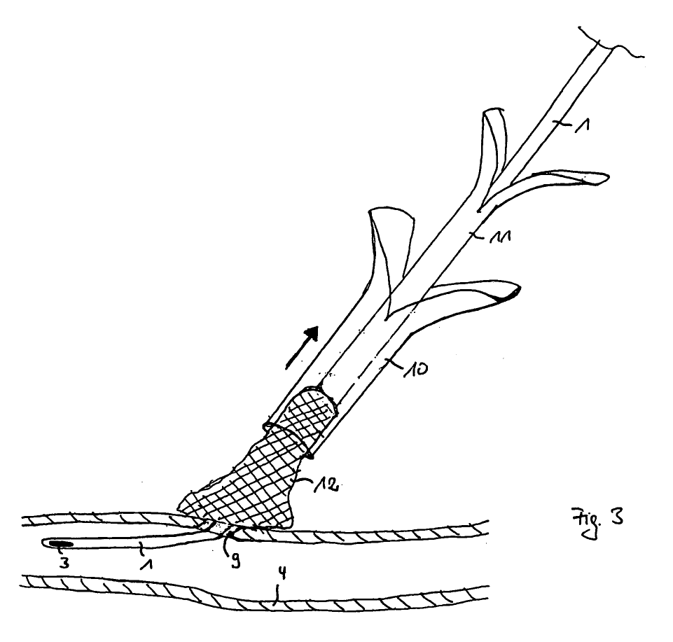

FIG. 3 shows the placement near the vessel wall of a hemostatic cuff by means of the ancillary elements;

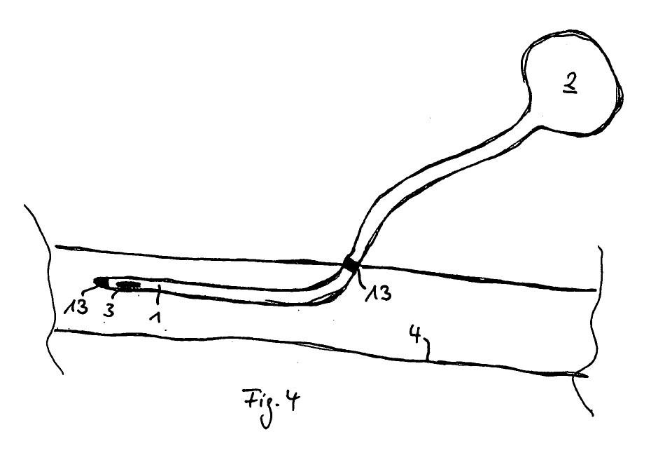

FIG. 4 is an overall view with position marks on the catheter;

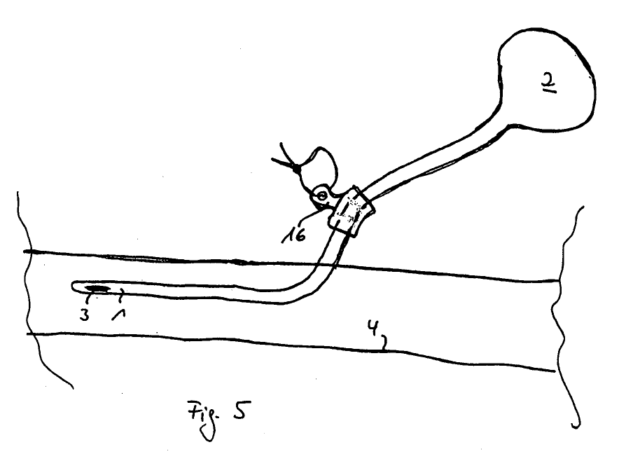

FIG. 5 is an overall view with a fixation clip on the catheter;

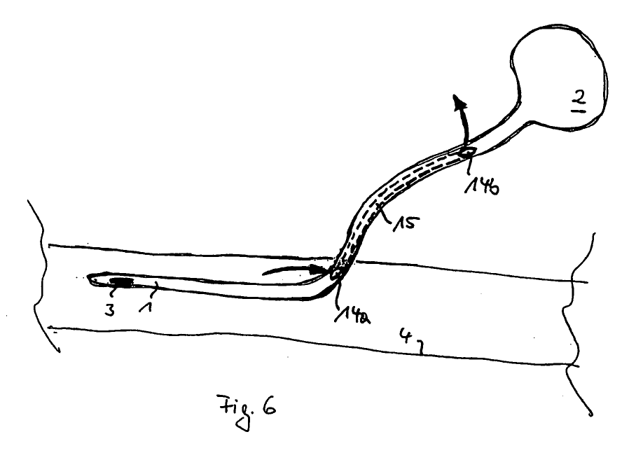

FIG. 6 is an overall view with a tubular guide wire within the catheter as a positioning aid.

The telemetric blood-pressure measuring system, which is illustrated in FIGS. 4, 5 and 6 in an overall view and various—also combinable—embodiments, is completely preassembled and encapsulated in a fluid-tight manner. The sensor catheter 1 and the transponder unit 2 here are wired to each other without a releasable connection.

This approach results in improved ease of handling, biocompatibility, and durability for the implant. The catheter 1 is inserted, i.e. implanted, along with its sensor-bearing tip 3 into a blood vessel 4, the connected transponder unit 2 preferably being implanted in the surrounding tissue, i.e. outside the blood vessel 4 in subcutaneous fatty tissue. By means of a reader, not shown here, measured variables from the blood vessel can be determined wirelessly from the transponder unit 2.

In a conventional puncture procedure for a blood vessel 4, a steel needle 5 is used to puncture the vessel 4, while a sheath 6 fitted over the steel needle 5 is inserted into the blood vessel 4. The needle 5 is then removed and the catheter 1 inserted through the horizontal sheath 6. After insertion of the catheter, the sheath 6 must be removed. For removal, sheaths are normally stripped off coaxially from catheter 1. The sheath is essentially a tube-like sleeve that coaxially surrounds the needle or other elements that are to be introduced into a blood vessel. Hereafter, the terms sheath/sleeve is used interchangeably.

In the preassembled system of FIGS. 4 through 6 used here, coaxial removal of the sheath is not possible since the transponder unit 2 is permanently attached to the catheter 1. Implantation of the sensor catheter 1 is therefore effected by a sheath 6 according to the invention that can be placed in the conventional manner as in FIG. 1 using the above-referenced needle 5 and that is separable at least singly, preferably doubly.

To this end, the sheath has at least one designed tear line, in particular, two opposing such tear lines, e.g. slits 7, running longitudinally, at least along part of its length, preferably over the entire length and beginning at its outer end 6a. After catheter placement and withdrawal from the vessel, the sleeve 6 can thus be split at least at one designed tear line 7 and peeled off the catheter 1, preferably into two halves 8. To ensure that complete peel-off capability (dividability over the entire length into two halves) is provided, PTFE can preferably be utilized as the material for such a sheath.

After puncture of the blood vessel 4 with the steel needle 5, insertion of the sleeve 6 into the vessel 4, removal of the steel needle 5, and introduction of the catheter 1 with sensor-bearing tip 3 several centimeters into the vessel 4 and withdrawal from the blood vessel 4 as well as stripping of the sheath 5, bleeding results, in particular from an arterial high-pressure vessel, since a puncture orifice 9 in the vessel wall matches the outer diameter of the removed sheath 6, yet is now occupied only by the inserted catheter 1 with a smaller outside diameter.

According to the invention, provision can be made whereby the bleeding is stopped, for example by advancing an additional sleeve 10 that is mounted coaxially on the catheter 1, in particular, preassembled, up to in front of the outer vessel wall. This can be seen in FIG. 2.

A further sleeve 11 serves as a retainer. It is preferably of smaller diameter than sleeve 10.

In an especially preferred embodiment, an inserted, in particular, pressed-in, e.g. sleeve-shaped cuff 12, composed for example of hemostatic material (hemostypticum, e.g. gelatin or collagen) can be provided within the sleeve 10 at the end of the sleeve facing the sensor. With the cuff 12, this may involve a pressed gelatin sponge.

Cuff 12 can be held in position by the retaining sleeve 11, while the sleeve 10 is retracted against fixed retaining sleeve 11, thereby releasing the cuff 12.

In response to release of the cuff 12, the cuff swells in front of the vessel wall. As a result, bleeding is stopped. Also during the peel-off procedure, the sleeve 10 can, for example, also be split into two parts and removed. In regard to dividability, the sleeve 10 in particular in two halves can have the same features as the sleeve 6 used to insert catheter 1.

Sleeve or sheath 11, which can also be peeled off and which can also be composed of PTFE, is preferably employed to retain the cuff 12 so as to release the cuff, additionally in an especially preferred manner be retained as an emergency sheath so that access to the vessel is maintained in the event the catheter system has to again be removed in the course of the procedure.

In an alternative approach to the described solution, a cuff permanently attached to the catheter shaft and preferably composed of swellable, in particular, hemostatic material, can be positioned in such a way by advancing the catheter that the cuff is located exactly at the entry point of the catheter 1 into the vessel wall, and thereby stops the bleeding. However, the disadvantage compared to the previously described solution is the fact that at least a small section of this cuff then can come into contact with the bloodstream inside the vessel, and can result in appositional thrombus formation and microembolisms due to the thrombogeneity of the hemostatic material.

In the event implantation of the catheter has not proceeded smoothly or the implant is defective, the preassembled sleeve 11 can function as an emergency sheath that can be advanced back into vessel 4, thereby maintaining access to the vessel. The entire procedure can thus be repeated as necessary. In regard to the peel-off capability, sleeve 11 can have these same features as were described for sleeve 6 and sleeve 10.

In order to ensure that the catheter 1 can be advanced sufficiently far into vessel 4 during radioscopic monitoring, a radio-opaque marker ring 13 can be provided at the tip 3, a few centimeters removed from this sensor-bearing tip 3 on the catheter shaft. FIG. 4 illustrates this. Catheter 1 can also be positioned with a second marker ring 13 in the proximity of the entrance to the vessel for the catheter.

Alternatively or additionally, a sharp bend with an angle of 30° to 90° in the catheter shaft can function as an additional check, including for positioning with ultrasonic monitoring. The vertex of this bend can be positioned in the vicinity of the vessel entrance of the catheter.

A small lateral orifice 14a in the catheter shaft, a few centimeters removed from the sensor-bearing tip, can also function as an alternative for ensuring correct positioning of the catheter. This lateral orifice 14a is connected through a passage 15 running inside the catheter to a side opening 14b in a portion near the transponder of the catheter 1 not intended for implantation into the vessel. FIG. 6 illustrates this. In response to exposure of the orifice 14a near the tip to the bloodstream inside the vessel, what results is a controlled reverse blood flow from the outer lateral orifice 14b, thereby providing an indication of the length of the section of catheter 1 located in the vessel. In order to stop this discharge of blood, the catheter can be retracted slightly so that tip-adjacent lateral orifice 14a no longer lies within the vessel lumen.

The above-described lateral orifice passage system 14/15 can also be utilized for the injection of radiographic or sonographic contrast media or other fluids into the blood vessel 4, e.g. in order to better image the blood vessel during the implantation procedure.

A preferably provided fixation clip 16 can serve to secure the catheter for a long term in an optimum position and prevent it from gradually sliding out of vessel 4, e.g. due to forces acting on the catheter section outside the vessel 4, the clip on the one hand fitting around the catheter shaft, and on the other hand having a securing element, e.g. a grommet or the like for suture fixation or clamp fixation to the adjacent tissue (e.g., muscle fascia). FIG. 5 illustrates this embodiment that is combinable with all of the other embodiments. The fixation clip is expediently located about 1 cm removed externally from the point of the vessel entrance for the catheter shaft, but may if necessary also be slid along the shaft.

In a preferred embodiment, the sensor can also be placed in the tip region 3 of the catheter 1 strictly laterally, that is, parallel to the longitudinal axis of the catheter so that during pressure measurement only static pressure is recorded, while the additive or subtractive measurement of back pressure is precluded.

In order to prevent long-term clotting (thrombus deposits) on the catheter portion located in the vessel 4, an antithrombogenic coating (e.g. heparin) is preferably applied to this catheter portion. An additional coating with cytostatic agents can prevent cell growth on the intravasal surface of the catheter or sensor.

Using currently available pressure sensors, the system can be employed, e.g. for long-term monitoring of blood pressure or for patients with hypertension or cardiac insufficiency. As soon as other sensors become available, other diseases can be measured as well: e.g., blood sugar levels in diabetes patients or oxygen/carbon dioxide partial pressures in patients with chronic lung diseases (e.g. COPD).

1. A device for measuring physiological measured variables inside the body, comprising a catheter having a sensor-bearing tip, the catheter being insertable into a blood vessel wherein the catheter is connected to a transponder unit to effect wireless transmission of at least one measured variable to an external reading station that after insertion of the catheter in a blood vessel is provided outside the blood vessel, the catheter and transponder being sealed in a fluid-tight manner.

2. The device according to claim 1 wherein at least some of the components required for insertion, in particular, sheaths and sleeves, are coaxially preassembled on the catheter.

3. The device according to claim 2 wherein the sheaths and/or sleeves have at least one desired tear line, in particular, at least one longitudinal slit at least at their outer end at a distance from the vessel, and by tearing off, in particular, peeling off in the longitudinal direction of the catheter, are removable from the catheter.

4. The device according to claim 1, further comprising
a preassembled cuff provided coaxially about the catheter shaft composed in particular of a hemostatic material, the cuff in particular being provided in a tearable first sleeve.

5. The device according to claim 1, further comprising
a second sleeve preassembled on the catheter shaft, in particular mounted coaxially as the last component on the transponder-adjacent section of the catheter shaft, in particular, that functions as a counter support for a cuff or as an emergency sheath that maintains vessel access.

6. The device according to claim 1 wherein on the catheter tip or on the catheter shaft at least one mark, in particular, radio-opaque points, rings, longitudinal threads or notches is provided that functions for orientation in regard to the length of the catheter implanted in the vessel.

7. The device according to claim 1 wherein a lateral orifice in the catheter adjacent the sensor is connected by a passage running within the catheter to a second transponder-adjacent lateral orifice in the catheter for the purpose of monitoring the catheter position or administering a contrast medium during the implantation procedure.

8. The device according to claim 1 wherein a fixation clip is provided on the catheter shaft that is engageable by a suture and/or a clamp, and by which the system is securable in the adjacent tissue, in particular, wherein the fixation clip is slidable on the catheter shaft.

9. The device according to claim 1 wherein the sensor is mounted laterally on the catheter, the sensor surface having an orientation parallel to the catheter axis.

10. The device according to claim 1 characterized in that wherein at least one catheter section located in the vessel has an antithrombogenic and/or cytostatic coating.

11. In combination with a blood catheter or needle, a sheath/sleeve coaxially fitted over the needle or catheter, the sheath/sleeve being dividable at least once longitudinally.

12. The sheath/sleeve according to claim 11 wherein it has at least one designed tear line running longitudinally, at least along part of its entire length beginning at its outer end.

13. The sheath/sleeve according to one of the foregoing claims 11 wherein it comprises a sleeve-shaped cuff by which an element provided for the insertion into a blood vessel is coaxially enveloped.

14. A blood catheter assembly comprising:
an elongated catheter insertable into a blood vessel and having a pair of ends;
a sensor at one of the ends;
a transponder at the other of the ends including means for wireless transmission; and
a fluid-tight membrane surrounding the entire catheter, sensor, and transponder such that the entire assembly can be fitted to a vein and installed subdermally.

15. The blood catheter assembly defined in claim 14, further comprising:
a sleeve surrounding and extending longitudinally along the catheter between the transponder and the tip, the sleeve being formed with a full-length longitudinal tear line.

16. The blood catheter assembly defined in claim 15, further comprising:
a second sleeve surrounding and extending longitudinally along the catheter outside the first-mentioned sleeve, the second sleeve also being formed with a full-length longitudinal tear line; and
a mass of hemostatic agent between the second sleeve and first sleeve.

17. The blood catheter assembly defined in claim 14 wherein the sleeve is formed with a pair of such longitudinal tear lines.

18. The blood catheter assembly defined in claim 14 wherein the transponder is an RFID device.

19. The blood catheter assembly defined in claim 14, further comprising:
a tube extending along a partial length of the catheter between its ends and having a front end opening outside the catheter immediately adjacent the tip and a rear end opening outside the catheter.

20. The blood catheter assembly defined in claim 14, further comprising:
a radiographically identical mark on the catheter between the ends.
